(19) United States
(12) Patent Application Publication
(10) Pub. No.: US 2025/0248740 A1
(43) Pub. Date: Aug. 7, 2025

(54) INTRAOSSEOUS TROCAR HANDLE

(71) Applicant: Concise Engineering, Inc., Clearwater, FL (US)

(72) Inventors: Justin Bushko, Oldsmar, FL (US); Kevin Quintero, Safety Harbor, FL (US); Daniel Riveros, Reddington Shores, FL (US)

(21) Appl. No.: 19/185,931

(22) Filed: Apr. 22, 2025

Related U.S. Application Data
(63) Continuation-in-part of application No. 29/914,827, filed on Oct. 23, 2023.

(51) Int. Cl. A61B 17/34

(52) U.S. Cl. CPC A61B 17/3472; A61B 17/3403; A61B 2017/3405

(57) ABSTRACT

The present disclosure provides trocar handles configured to enable convenient manual (e.g., motorless) advancement of intraosseous needles into bone of a subject.

### PRIORITY CLAIM

Is application claims priority to U.S. Provisional Patent Application Ser. No. 63/592,296, filed on Oct. 23, 2023, and is a continuation-in-part application of U.S. Design patent application Ser. No. 29/914,827, filed on Oct. 23, 2023, the entire contents of each of which are incorporated herein by reference and relied upon.

### FIELD

The present disclosure provides trocar handles, especially for manual or non-powered use, for example for use with bone marrow harvesting or biopsy needles.

### BACKGROUND

Accurate and secure placement of intraosseous needles in a subject's bone can be a risky and painful process. Recently, powered drill-type drivers have been developed to drive intraosseous needles into the bone. But such devices require nearby a power supply or maintaining charge of a battery power source. Powered drivers are bulky and offer clinicians limited ability to correct the angle or power at which the intraosseous needle is advanced at and through the bone.

A need remains for trocar handles that can be used to advance an intraosseous needle through a subject's bone tissue without requiring a power source, such as a powered drill or other electrical device.

### SUMMARY

In some embodiments, the present disclosure provides a trocar handle 10 for coupling with an intraosseous needle assembly N, the trocar handle comprising: a handle portion 100 comprising: a grip portion 130 disposed at a proximal handle end 110, a neck portion 140 disposed at a distal handle end 120; and a needle chuck portion 200 comprising: a drive bit 230 disposed at a proximal needle chuck end 210, and a clasp 255 disposed at a distal needle chuck end 220.

In other embodiments, the present disclosure provides a trocar handle 10 for use with an intraosseous needle assembly N, the trocar handle comprising: a handle portion 100 comprising: a grip portion 130 disposed at a proximal handle end 110 and including: a handle lattice 134, a handle surface 132 surrounding the handle lattice 134, and a neck portion 140 disposed at a distal handle end 120 and including a handle lattice 134; and a needle chuck portion 200 comprising: a drive bit 230 disposed at a proximal needle chuck end 210 and colinear with a center axis 151 of the trocar handle, a drive plate 232 disposed proximal to the drive bit and configured to contact a proximal portion N100 of the intraosseous needle assembly, a clasp 255 disposed at a distal needle chuck end 220 and including at least one feature configured to removably mate with a complementary feature of a neck portion N200 of the intraosseous needle assembly, and a chuck arm 250 separating the clasp from the drive bit.

In other embodiments, the present disclosure provides a trocar handle for coupling with an intraosseous needle assembly, the trocar handle comprising: a handle portion comprising: a grip portion disposed at a proximal handle end, a neck portion disposed at a distal handle end; and a needle chuck portion associated with the neck portion and comprising: a drive bit disposed at a proximal needle chuck end, and a needle securing cup disposed at the distal handle end and configured to reduce unintended disengagement of the intraosseous needle assembly from the trocar handle.

In still other embodiments, the present disclosure provides a method 20 of advancing an intraosseous needle assembly N into a bone of a subject, the method comprising: mating an intraosseous needle assembly into a needle chuck portion 200 of a trocar handle 10 of any one preceding claim; contacting a needle portion N300 of the intraosseous needle assembly with a surface of a bone of the subject; grasping the handle portion 100 of the trocar handle with a palm of a hand; applying pressure from the hand towards the bone of the subject substantially along the centerline CL of the trocar handle to advance the needle portion into the bone; and unmating the intraosseous needle assembly from the needle chuck after the needle portion has advanced a desired amount into the bone.

### OBJECTS OF THE DISCLOSED TECHNOLOGIES

It is one object of the disclosed technologies to provide a manual (e.g., non-electrical, non-powered) handle for advancing an intraosseous needle into bone of a subject.

It is another object of the disclosed technologies to provide an ergonomically comfortable manual (e.g., non-electrical, non-powered) handle compatible with intraosseous needles that are configured to be driven into bone of a subject by an electrically powered driver.

It is another object of the disclosed technologies to provide a manual (e.g., non-electrical, non-powered) handle for driving intraosseous needle, wherein the handle has a reduced weight without reduction in rigidity or structural integrity compared to a comparable solid or hollow handle.

### BRIEF DESCRIPTION OF THE FIGURES

FIG. 1 shows a front view of a trocar handle consistent with one embodiment of the present disclosure.

FIG. 2 shows an intraosseous needle assembly mated with the trocar handle of FIG. 1.

FIG. 3 shows a side view of the trocar handle of FIG. 1.

FIG. 4 shows a front perspective view of the trocar handle of FIG. 1.

FIG. 5 shows a bottom perspective view of the trocar handle of FIG. 1.

FIG. 6 shows a side perspective view of the trocar handle of FIG. 1

FIG. 7 shows an enlarged perspective view of the needle chuck portion of the trocar handle of FIG. 1.

FIG. 8 shows a top perspective view of a trocar handle consistent with a second embodiment of the present disclosure.

FIG. 9 shows a bottom perspective view of the trocar handle of FIG. 8.

FIG. 10 shows a front view of the trocar handle of FIG. 8.

FIG. 11 shows a left side view of the trocar handle of FIG. 8.

FIG. 12 illustrates stepwise use of the trocar handle of FIG. 8 to remove a cannula from bone of a subject, consistent with one embodiment of the present disclosure.

FIG. 13 is a flow diagram illustrating a method of using a trocar handle consistent with the present disclosure to advance an intraosseous needle into a subject's bone according to one embodiment.

These and other embodiments are disclosed in further detail below.

### DETAILED DESCRIPTION

Trocar handles consistent with the present disclosure generally do not include a motor or any other powered element, but rather enable a user (e.g., clinician) to conveniently and efficiently advance an intraosseous needle into a subject's bone. Trocar handles consistent with the present disclosure are particularly convenient for use in settings without power sources, or where carrying a specialized power tool would be inconvenient.

Generally, a trocar handle 10 consistent with the present disclosure include a handle portion 100 and a needle chuck portion 200 disposed at a distal end of the handle portion 100.

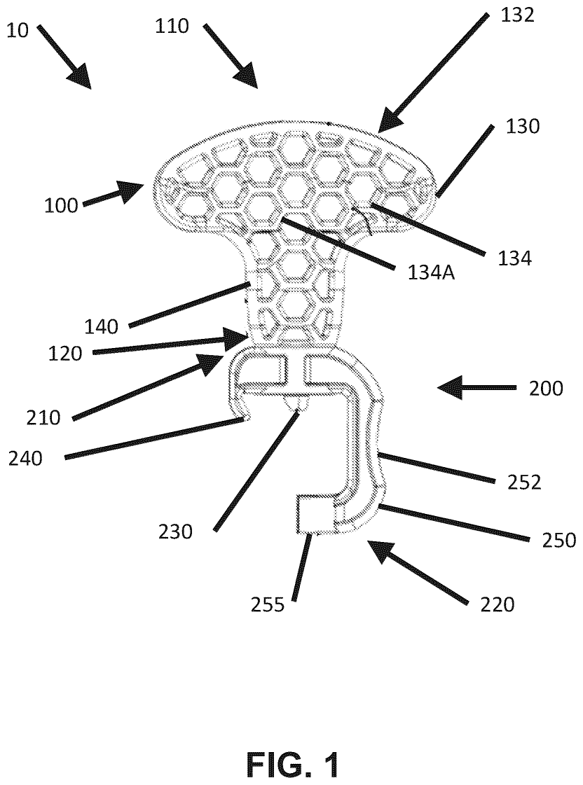

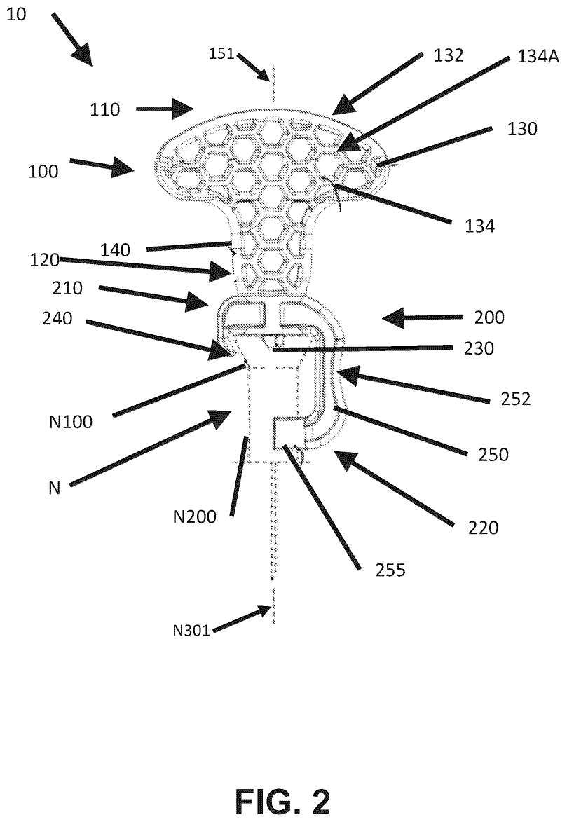

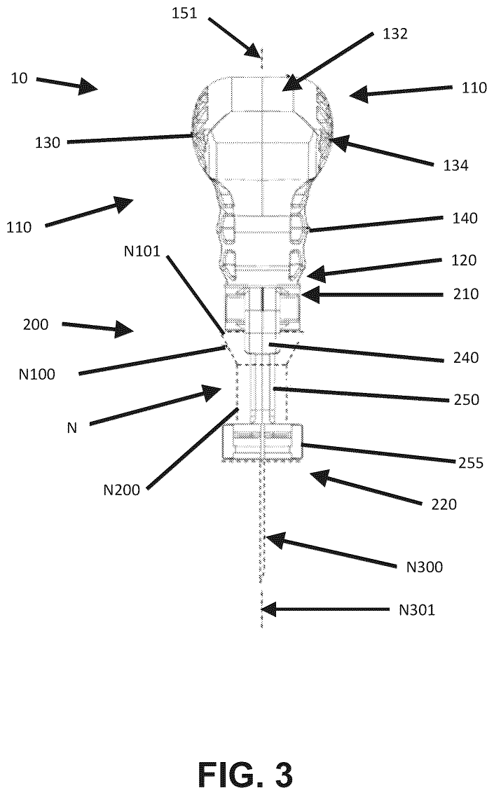

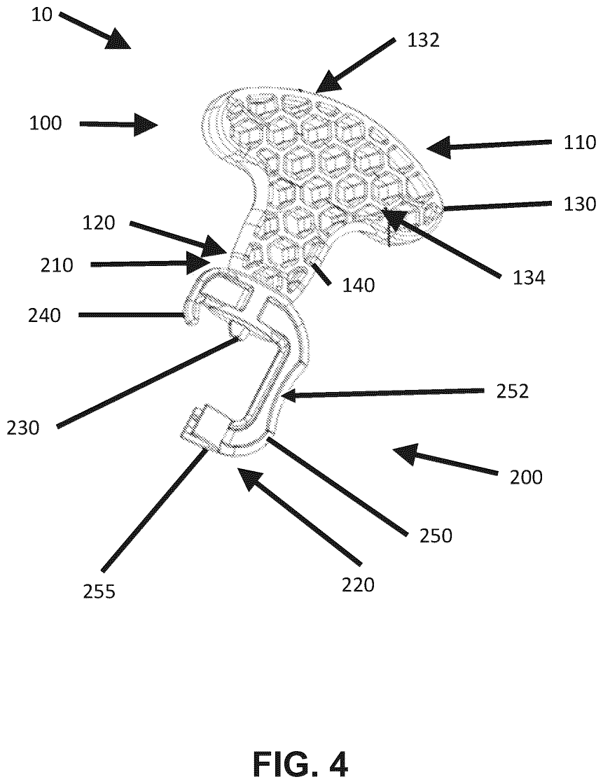

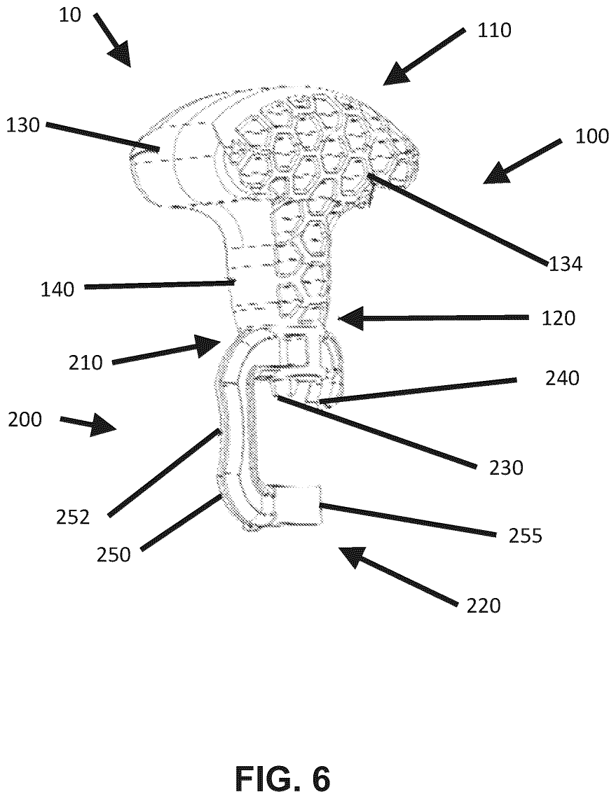

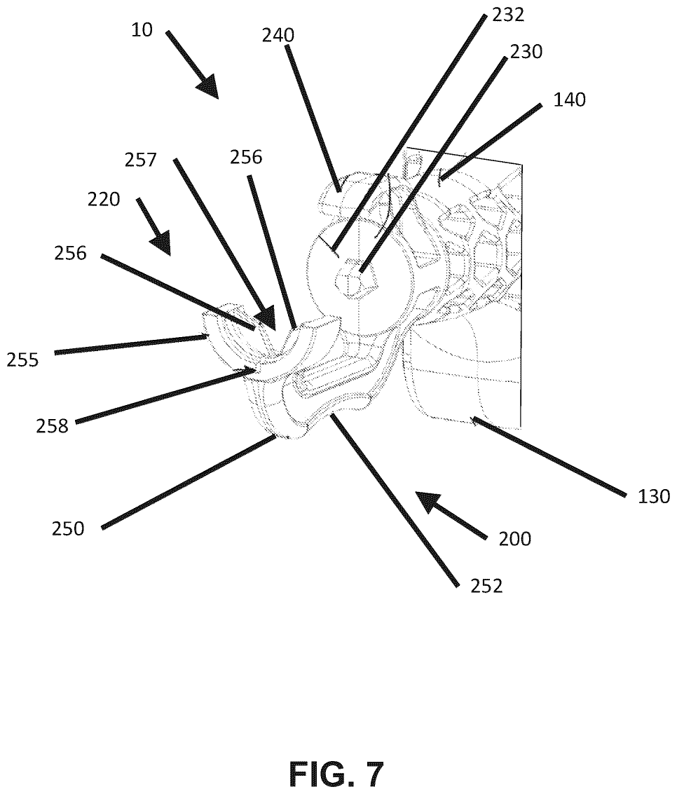

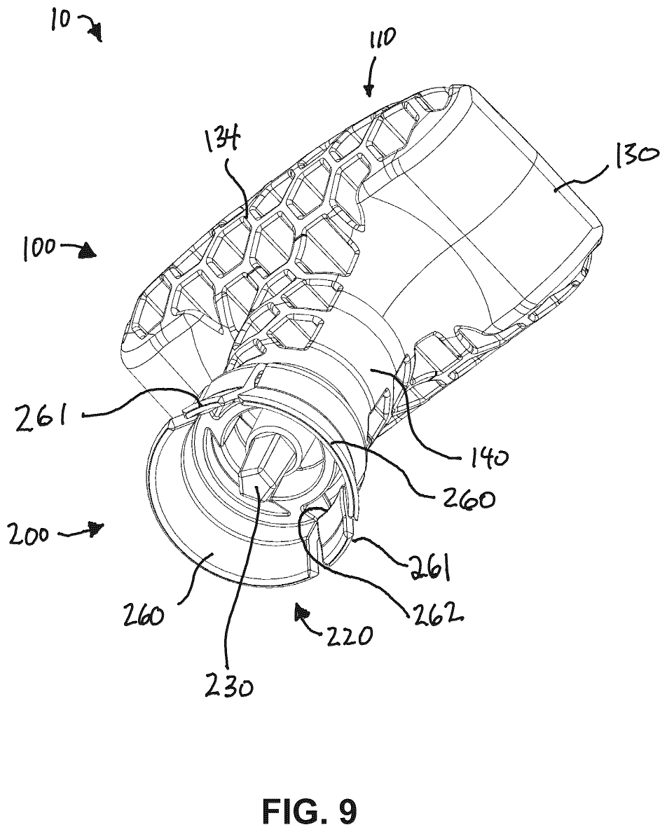

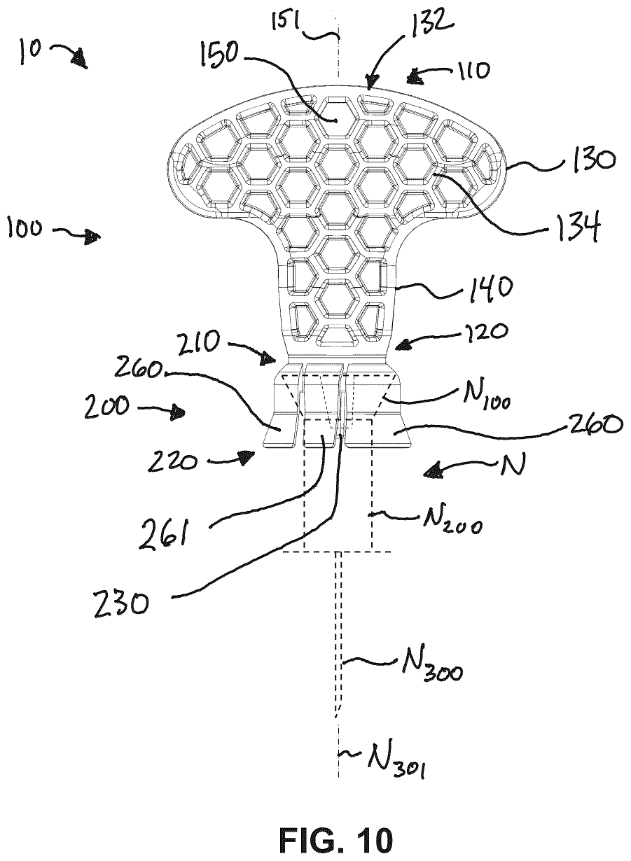

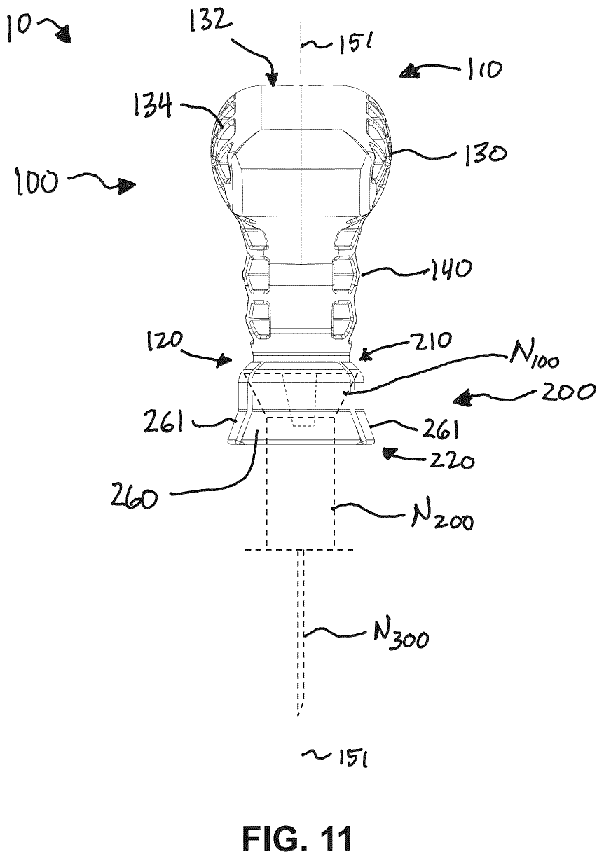

Referring now to FIGS. 1-11, a trocar handle 10 consistent with the present disclosure includes a handle portion 100, and a needle chuck portion 200. The handle portion 100 is used by a user to support the needle chuck portion 200 during insertion of a needle into a subject. The needle chuck portion 200 supports an intraosseous needle assembly N (best shown in FIG. 2) during insertion and is rigidly attached to the handle portion 100 at the proximal needle chuck end 210.

The handle portion 100 includes a proximal handle end 110. The proximal handle end 110 provides an interface for a user (e.g., a user's hand). The handle surface 132 surrounds the handle lattice 134 and is used by a user to apply force to the needle chuck portion 200 (e.g., in the distal direction towards a subject's bone). The handle portion 100 may include a grip portion 130 configured to provide a user with a stable structure for applying force (and torque about the center axis 151) to the needle chuck portion 200, and reducing slippage between the trocar handle 10 and the user's hand. The handle lattice 134 may also provide structural support for transferring a force from the proximal handle end 110 to the needle chuck portion 200. In some embodiments, the handle lattice 134 provides the trocar handle 10 with a reduced weight compared to a trocar handle that does not include a handle lattice (e.g., compared to a comparable trocar handle including a solid handle portion). In some embodiments, the handle lattice 134 provides the trocar handle 10 with increased rigidity compared to a comparable trocar handle that does not include a handle lattice (e.g., a comparable trocar handle that includes a hollow handle portion).

The handle lattice 134 may provide structure to stabilize the device during use. For example, in some embodiments the handle lattice 134 includes fins 134A configured to provide an expanded volume (e.g., compared to a comparable trocar handle of similar weight that does not include a handle lattice) for an ergonomically stable interface with a user's hand.

In some embodiments, the handle portion 100 includes a neck portion 140 distal to the grip portion 130 and proximal to the needle chuck portion 200. In some embodiments, the neck portion 140 includes handle lattice 134. In other embodiments, the neck portion 140 does not include handle lattice 134 but is instead solid or hollow. The neck portion 140 is rigidly attached at the distal handle end 120 to the needle chuck portion 200 at the proximal needle chuck end 210. The neck portion 140 may have a narrower dimension (e.g., smaller volume) than the grip portion 130 so that the neck portion 140 may protrude between a user's fingers comfortably. This may allow the user to firmly grip the grip portion and maintain control of the device 10 during use.

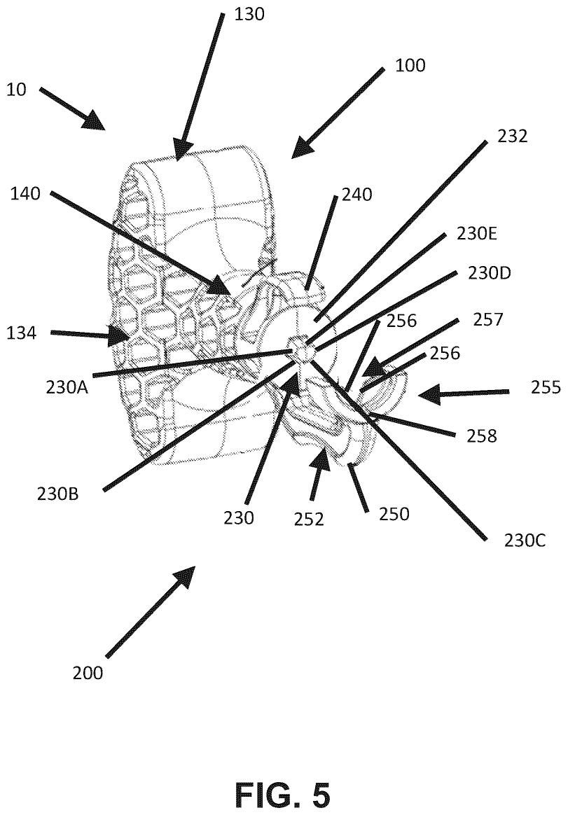

The needle chuck portion 200 support an intraosseous needle assembly N (best shown in FIG. 2) during use. In some embodiments, the center axis 151 aligns with the handle portion 110 and the drive bit 230. A drive bit 230 may be included at the proximal needle chuck end 210 of the needle chuck portion 200. The drive bit 230 may engage a drive pocket included on the intraosseous needle assembly N at the proximal portion N₁₀₀. The drive bit 230 may or may not align with the center axis 151. The drive bit 230 may protrude from drive plate 232 (best shown in FIG. 5). The drive plate 232 may be configured to support the proximal portion N100 of the intraosseous needle assembly N. During use, a force may be transferred from the drive plate 232 to the proximal portion N₁₀₀ of the intraosseous needle assembly N. The drive bit 230 may include features for twisting the intraosseous needle assembly N during insertion into bone.

In some embodiments, the drive bit 230 includes chuck torque faces 230A, 230B, 230C, 230D, and 230E (best shown in FIG. 5). The chuck torque faces 230A, 230B, 230C, 230D, and 230E may engage complementary features on the proximal portion N₁₀₀ of the intraosseous needle assembly N. During insertion of the needle N₃₀₀ a user may twist the handle portion 100 and transfer torque from the chuck torque faces 230A, 230B, 230C, 230D, and 230E to the intraosseous needle assembly N. The needle chuck portion 200 may include additional features for stabilizing the intraosseous needle assembly N.

In some embodiments, the needle chuck portion 200 may include a proximal tab 240 configured for retaining the intraosseous needle assembly N. The proximal tab 240 is configured to improve retention of the intraosseous needle assembly N in the needle chuck portion 200 and is located at the proximal needle chuck end 210 of the needle chuck portion 200. In some embodiments, the proximal tab 240 slips over a needle retaining feature N101 for supporting the proximal portion N100. The proximal tab 240 may be any feature that interfaces with the needle retaining feature N101 and is not limited to a cantilever beam (e.g. a protrusion inserted into a needle feature may be used as well as snap features). In some embodiments, the proximal tab 240 may provide for needle engagement with the drive bit 230 such as deflecting during placement of the intraosseous needle assembly N into the proximal needle chuck end 210. In some embodiments, the proximal tab 240 may provide for engagement of the drive plate 232 with the proximal portion N₁₀₀ of the intraosseous needle assembly N. Proximal needle chuck end 210 may include additional retaining features.

In some embodiments, the intraosseous needle assembly N may be further supported by a clasp 255. The clasp 255 may provide retention of the intraosseous needle assembly N and may also align a needle center axis N₃₀₁ with the center axis 151. This alignment may provide a known location for a user to apply force during insertion of the needle N₃₀₀ thus safeguarding the insertion process and providing a more ergonomic process. The clasp 255 may or may not align with the drive bit 230. The clasp 255 may be positioned by additional needle chuck portion 200 features and include additional features.

The needle chuck portion 200 may include a chuck arm 250 extending from the proximal needle chuck end 210 to the distal needle chuck end 220 and configured to support the clasp 255. The clasp 255 may include clasp tabs 256 which engage features on the intraosseous needle assembly N for retaining the needle neck portion N200 of the intraosseous needle assembly N. In some embodiments, the clasp 255 includes a slot 258 configured to determine the stiffness of the clasp tabs 256. This stiffness determines the flexibility of the clasp 255 against the needle neck portion N₂₀₀ of the intraosseous needle assembly N. In some embodiments the chuck arm 250 may include a curved portion 252 configured to increase flexibility of the chuck arm 250. In some embodiments, the clasp 255 may include at least one tab gap 257 configured to allow removal and insertion of the intraosseous needle assembly N. In some embodiments the tab gap 257 may be located between two clasp tabs 256. In others, the tab gap 257 may be a simple hole or other feature which allows for removal of the intraosseous needle assembly N.

Disclosed herein is a trocar handle 10 for coupling with an intraosseous needle assembly N, the trocar handle comprising a handle portion 100. The handle portion 100 having a grip portion 130 disposed at a proximal handle end 110, a neck portion 140 disposed at a proximal handle end 110, the neck portion 140 disposed at a distal handle end 120. The trocar handle 10 includes a needle chuck portion 200. The needle chuck portion comprising a drive bit 230 disposed at a proximal needle chuck end 210, and a clasp 255 disposed at a distal needle chuck end 220. In some embodiments, drive bit 230 may include one or a plurality of chuck torque faces 230A, 230B, 230C, 230D, and 230E. In some embodiments, the trocar handle 10 further comprises a chuck arm 250 extending from the proximal needle chuck end 210 to the distal needle chuck end 220.

In some embodiments, the trocar handle 10 further comprises a proximal tab 240 disposed at the proximal needle chuck end 210 and adjacent the drive bit 230 and configured to improve retention of the intraosseous needle assembly N in the needle chuck portion 200. In some embodiments the grip portion 130 includes a handle lattice 134 substantially surrounded or surrounded by a handle surface 132. In some cases, clasp 255 is configured to removably mate with a needle neck portion N200 of the intraosseous needle assembly N.

The clasp 255 may include a slot 258 configured to increase flexibility of the clasp 255. The clasp 255 may include at least one tab 256 and at least one gap 257. The gap 257 may be configured to removably mate with and retain at least a portion of the intraosseous needle assembly N.

In some embodiments, the proximal needle chuck end 210 may include a drive plate 232 configured to contact a proximal portion (N100) of the intraosseous needle assembly N. Additionally, the chuck arm 250 may include an arm curve 252 configured to increase flexibility of the chuck arm 250. This flexibility allows for the proximal portion N100 of the intraosseous needle assembly N to fully contact the drive plate 232 during insertion into a bone.

In some embodiments, ergonomics may be added to aid the user. As an example and without limitation, the handle surface 132 may be configured to ergonomically mate with a hand (e.g., palm) of a user. As another example and without limitation, the drive bit and the handle portion are substantially colinear with a center axis 151 of the trocar handle 10. An additional example is a handle portion having a center axis 151 that is colinear with a needle center axis N301.

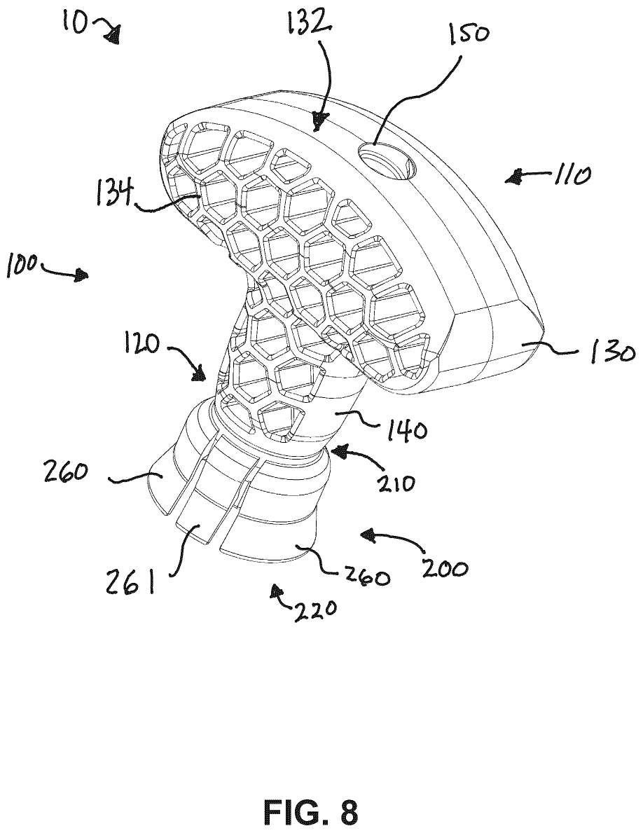

Referring now specifically to FIGS. 8-12, a trocar handle 10 consistent with some embodiments of the present disclosure may include a threaded cavity 150 configured to mate with a threaded portion of a needle N that has already been driven into bone of a subject. The threaded cavity 150 may include threads about its internal surface that complement threads on the outer surface of the needle N. Twisting the trocar handle 10 when the threaded cavity 150 and the threads of the needle N are aligned causes the needle N to seat within the threaded cavity 150. The trocar handle 10 may then be used to withdraw the needle N from the subject's bone with relatively more leverage compared to withdrawing the needle N by directly grasping the needle N (e.g., using the clinician's hands or other tools).

In some embodiments, including those generally consistent with FIGS. 8-12, the needle chuck portion 200 may include a needle insertion cup 260 disposed about the drive bit 230. When present, the needle insertion cup 260 enhances stability of the needle N when it is engaged with the drive bit 230. For example and without limitation, the needle insertion cup 260, when present, reduces or eliminates the risk that the needle N unintentionally disengages from the trocar handle 10 due to gravitational forces or unintended jostling or bumping.

In some embodiments, the needle insertion cup 260 includes one or more (e.g., two, three, four, or five) needle securing tabs 261. The needle securing tabs 261 are relatively more flexible than the remainder of the needle insertion cup 260 and generally include a ridge 262 proximal to the drive bit 230 that is configured to engage the outer edge of the needle N. When present, the one or more needle securing tabs 261 retain (e.g., temporarily retain) the needle N in engagement with the drive bit 230 to, for example, prevent the needle N from falling away from the trocar handle 10 due to gravity or unintended jostling or bumping. The needle securing tabs 261 are configured to cause the needle N to disengage from the drive bit 230 when desired by, for example, pivoting the proximal handle end 110 relative to the vertical axis of the needle N.

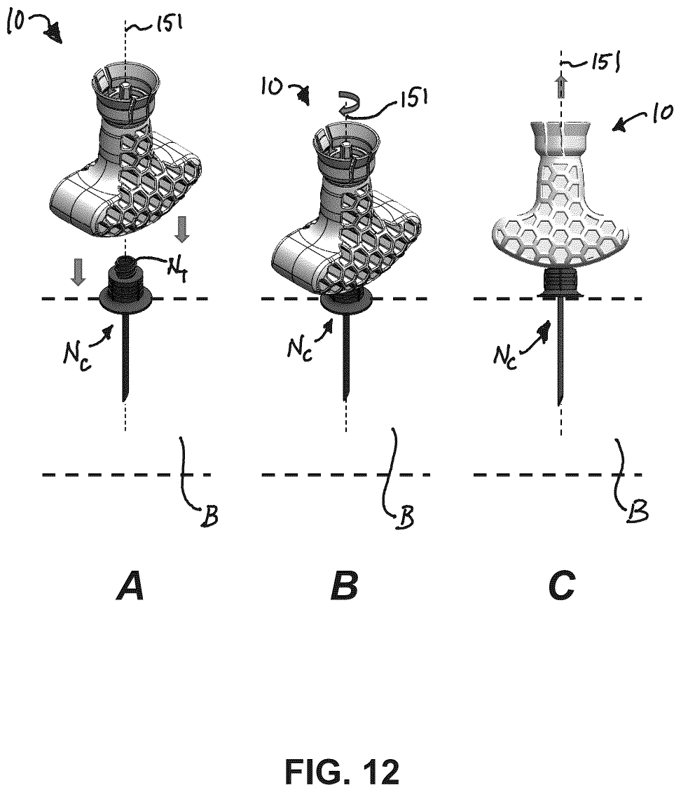

Referring now to FIG. 12, embodiments that include a threaded cavity 150, including those generally consistent with FIGS. 8-11, may be used to conveniently remove a cannula portion N_{C} of the needle N from a subject's bone B. First (step A), the threaded cavity 150 of the trocar handle 10 is lined up with the threaded portion N_{T} of the cannula N_{C}. Rotation (step B) of the trocar handle 10 about its central axis 151 engages the trocar handle 10 with the cannula portion N_{C}. Once engaged, the trocar handle 10 can be pulled (step C) away from the bone B to withdraw the cannula portion N_{C} from the bone B without the need for electrically powered devices.

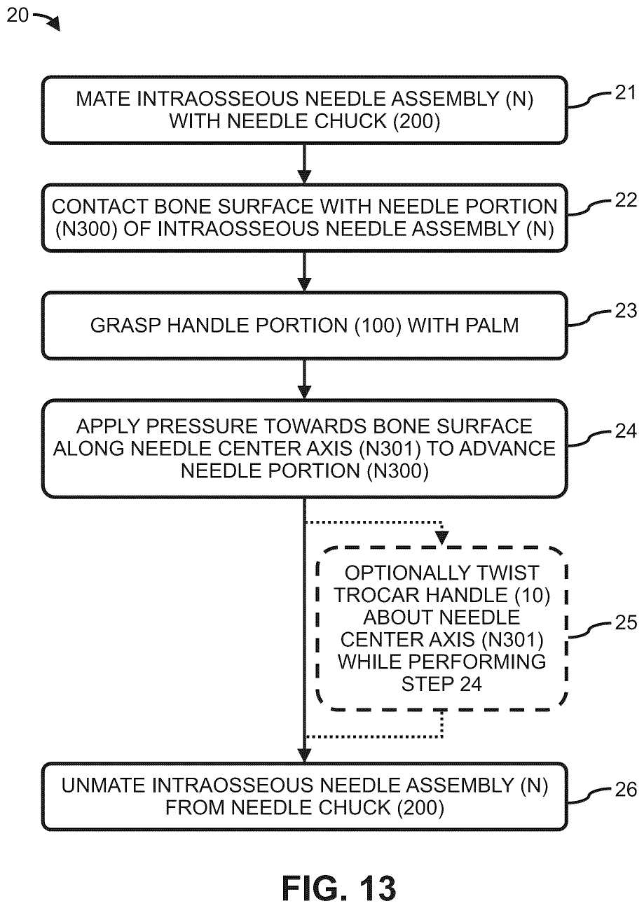

Referring now to FIG. 13, the present disclosure provides methods 20 of using a trocar handle 10 as described herein to advance an intraosseous needle N into a subject's bone. Generally, the method 20 does not include a step of advancing the intraosseous needle assembly N into the subject's bone using electrical power (e.g., a motor).

In some embodiments, the method 20 includes a step 21 of mating the intraosseous needle assembly N into the needle chuck portion 200 of the trocar handle 10. In some embodiments, the needle chuck portion 200 mates with the neck portion N₃₀₀ of the intraosseous needle assembly N. For example and without limitation, in some embodiments the step 21 of mating comprises contacting one or more mating features 256, 257 of the clasp 255 with a complementary feature of a neck portion N₂₀₀ of the intraosseous needle assembly N.

In some embodiments, the method 20 includes a step 22 of contacting the surface of the subject's bone with the needle portion N₃₀₀ of the intraosseous needle assembly N. The step 22 of contacting the surface of the subject's bone with the needle portion N₃₀₀ of the intraosseous needle assembly N may in some embodiments occur after the step 21 of mating the intraosseous needle assembly N into the needle chuck portion 200 of the trocar handle 10. In some embodiments, a layer of skin or other tissue is disposed over the subject's bone; the step 22 of contacting may thus also include a step of applying a numbing agent (e.g., lidocaine) to the subject's skin at or near the desired location for penetrating the subject's bone before contacting the surface of the subject's bone with the needle portion N₃₀₀ of the intraosseous needle assembly N.

In some embodiments, the method 20 includes a step 23 of grasping the handle portion 100 of the trocar handle with a palm of the user's (e.g., clinician's) hand. The step 23 of grasping the handle portion 100 of the trocar handle with a palm of the user's (e.g., clinician's) hand may in some embodiments occur after the step 22 of contacting the surface of the subject's bone with the needle portion N₃₀₀ of the intraosseous needle assembly N. In other embodiments, the step 23 of grasping the handle portion 100 of the trocar handle with a palm of the user's (e.g., clinician's) hand may first occur before the step 22 of contacting the surface of the subject's bone with the needle portion N₃₀₀ of the intraosseous needle assembly N, and may also occur after the step 22 of contacting the surface of the subject's bone with the needle portion N₃₀₀ of the intraosseous needle assembly N. Typically, the user (e.g., clinician) will perform this step 23 using his or her dominant hand.

In some embodiments, the method 20 includes a step 24 of applying pressure from the hand towards the bone of the subject substantially along the center axis 151 of the trocar handle 10 to advance the needle portion N₃₀₀ into the subject's bone. In some embodiments, the step 24 of applying pressure occurs after the step 23 of grasping the handle portion 100 of the trocar handle with a palm of the user's (e.g., clinician's) hand. Applying pressure not substantially along the center axis 151 is disfavored in this method 20 because doing so will increase the likelihood that the needle portion N₃₀₀ will slide along the bone surface rather than penetrate through the bone surface and into the bone's medullary cavity. In some embodiments, this step 24 of applying pressure comprises placing the non-dominant hand of the user (e.g., clinician) over the back side of the user's dominant hand to provide additional pressure and stabilization of force substantially colinear with the center axis 151.

In some embodiments, the method 20 includes a step 25 of twisting the trocar handle 10 about the center axis 151 while performing the step 24 of applying pressure from the hand towards the bone of the subject substantially along the center axis 151 of the trocar handle 10 to advance the needle portion N₃₀₀ into the subject's bone. Without wishing to be bound by theory, it is presently believed that twisting the trocar handle 10 imparts a twisting motion to the tip of the needle portion N₃₀₀ of the intraosseous needle assembly N, causing the beveled tip edges of the needle portion N₃₀₀ to gradually remove small bits of bone to improve boring action.

In some embodiments, the method 20 includes a step 26 of unmating the intraosseous needle assembly N from the needle chuck portion 200 after the needle portion N₃₀₀ has advanced a desired amount into the subject's bone. Generally, step 26 is performed after the step 24 of applying pressure from the hand towards the bone of the subject substantially along the center axis 151 of the trocar handle 10 to advance the needle portion N₃₀₀ into the subject's bone (with or without performance of optional step 25) has been completed. In some embodiments, the needle portion N₃₀₀ includes graduation markings enabling the user (e.g., clinician) to conveniently determine the depth into which the needle portion N300 has penetrated into the subject's bone. After step 26 has been successfully completed, the distal tip of the needle portion N₃₀₀ will be disposed in the medullary cavity of the subject's bone, and the proximal end of the needle portion's lumen will be available for connection to medical tubing or other fluid delivery or collection apparatus. In some embodiments, for example in which the intraosseous needle assembly N is an Arrow® EZ-IO® needle assembly (Teleflex® Incorporated), the step 26 of unmating the intraosseous needle assembly N from the needle chuck portion 200 may additionally include separating a stylet portion of the intraosseous needle assembly N from the needle portion N₃₀₀ to expose the distal end of the needle portion N₃₀₀.

1-21. (canceled)

22. A trocar handle for coupling with an intraosseous needle assembly, the trocar handle comprising:
a handle portion comprising:
a grip portion disposed at a proximal handle end,
a neck portion disposed at a distal handle end; and
a needle chuck portion associated with the neck portion and comprising:
a drive bit disposed at a proximal needle chuck end, and
a needle securing cup disposed at the distal handle end and configured to reduce unintended disengagement of the intraosseous needle assembly from the trocar handle.

23. The trocar handle of claim 22, wherein the needle securing cup includes one or more needle securing tabs configured to retain the intraosseous needle assembly within the needle securing cup.

24. The trocar handle of claim 23, wherein the one or more needle securing tabs are configured to disengage from the intraosseous needle assembly after the intraosseous needle assembly has been inserted into bone of a subject.

25. The trocar handle of claim 22, wherein the handle portion further comprises a threaded cavity configured to mate with a threaded portion of the intraosseous needle assembly.

26. The trocar handle of claim 22 further comprising a proximal tab disposed at the proximal needle chuck end and adjacent the drive bit, and configured to improve retention of the intraosseous needle assembly in the needle chuck portion.

27. The trocar handle of claim 22, wherein the grip portion includes a handle lattice substantially surrounded or surrounded by a handle surface.

28. The trocar handle of claim 22, wherein the proximal needle chuck end further comprises a drive plate configured to contact a proximal portion of the intraosseous needle assembly.

29. The trocar handle of claim 22, wherein the handle surface is configured to ergonomically mate with a hand of a user.

30. The trocar handle of claim 22 any one of claims 22-29, wherein the drive bit and the handle portion are substantially colinear with a center axis of the trocar handle.

31. The trocar handle of claim 22, wherein the drive bit comprises at least one chuck torque face.

32. A method of advancing an intraosseous needle assembly into a bone of a subject, the method comprising:
mating an intraosseous needle assembly into a needle chuck portion of a trocar handle of any one preceding claim;
contacting a needle portion of the intraosseous needle assembly with a surface of a bone of the subject;
grasping the handle portion of the trocar handle with a palm of a hand;
applying pressure from the hand towards the bone of the subject substantially along the center axis of the trocar handle to advance the needle portion into the bone; and
unmating the intraosseous needle assembly from the needle chuck after the needle portion has advanced a desired amount into the bone.

33. The method of claim 32, wherein the step of applying pressure further comprises twisting the trocar handle about the center axis.

34. The method of claim 32 further comprising:
mating a threaded cavity of the trocar handle with a threaded portion of the intraosseous needle assembly that is exposed from the bone to form a trocar handle-embedded needle assembly; and
withdrawing the intraosseous needle assembly from the bone by pulling, away from the bone, the trocar handle-embedded needle assembly formed in the step of mating the threaded cavity.
